(19) United States
(12) Patent Application Publication
(10) Pub. No.: US 2007/0295398 A1
(43) Pub. Date: Dec. 27, 2007

(54) Conducting Polymers With Porphyrin Cross-Linkers

(76) Inventors: Chee On Too, Farmborough Heights (AU); Jun Chen, Wollongong (AU); Gordon Wallace, Gwynnevelle (AU); David L. Officer, Palmerston North (NZ); Mason Wayne Campbell, Palmerston North (NZ); Anthony Keiran Burrell, Los Alamos, NM (US); Errol Gavin Collis, Los Alamos, NM (US)

Correspondence Address: KENYON & KENYON LLP, ONE BROADWAY, NEW YORK, NY, 10004, US

(21) Appl. No.: 10/544,085

(22) PCT Filed: Jan. 30, 2004
(86) PCT No.: PCT/AU04/00111

§ 371 Date: Feb. 9, 2007

(30) Foreign Application Priority Data
Jan. 31, 2003 (AU) 200300404

(51) Int. Cl. H01L 31/18; C07D 487/22; C08G 61/12

(52) U.S. Cl. 136/258; 252/521.6; 524/547; 526/241; 526/256; 526/258; 526/259; 526/270; 526/275; 526/284; 526/310; 526/347.1

(57) ABSTRACT

The invention relates to a cross-linking monomer having the structure Q-(L)ₙ-P-(L′)ₘ;-Q′, where Q and Q′ are polymerisable units, L and L′ are linkers providing direct or indirect electronic communication between Q and P and between P and Q′, and P is an electrofunctional unit, and also to polymers prepared from such monomers. Q for example may be a heteroaromatic ring such as thiophene, furan and pyrrole. The electrofunctional group may be, for example, porphyrin, substituted porphyrin, phthalocyanine or substituted phthalocyanine. The invention also relates to an electrofunctional material including a base material and a cross-linked polymer such as described.

### TECHNICAL FIELD

The present invention relates to improvements in conductive electrofunctional polymers, improvements in methods of synthesising such electrofunctional polymers, and the use of such polymers.

### BACKGROUND ART

Any discussion of the prior art throughout the specification should in no way be considered as an admission that such prior art is widely known or forms part of common general knowledge in the field.

Porphyrins are interesting molecular structures which provide the basis of the light harvesting capabilities of chlorophyll and the oxygen binding capabilities of heme in addition to possessing electron transfer mediation capabilities.

Porphyrins are one of a number of electrofunctional groups or units capable of participating in electron transfer.

The introduction of porphyrin groups into the structure of a polymer is intended to introduce the properties of the porphyrin into the polymer. These properties include metal binding, redox activity, photoactivity and light harvesting. Polymers exhibiting these properties can then be incorporated into or applied to the surfaces of devices. The devices can be used for a range of applications.

Procedures that enable porphyrin groups to be immobilised on or integrated in polymer chains are known. This may be achieved by:

(i) forming copolymers with vinyl monomers,

(ii) covalently binding porphyrins to preformed polymers, or

(iii) by polymerising groups (vinyl) attached to a natural or synthetic porphyrin.

Porphyrins have also simply been added to conducting polymer mixtures.

The synthesis and application of porphyrin containing polymers has been reviewed (Bao, Z.; Yu, L. Trends in Polymer Science, 1995, 3, 159 and references cited therein).

A particularly interesting process involves attachment of electropolymerisable groups to porphyrins. The products of this process can then be used to form a thin coating of the polymeric material on electrodes such as platinum or ITO glass.

Using porphyrin-containing monomers, insoluble films can be electrodeposited and the devices produced used for a range of applications, including chemical and bio-sensing, solar energy conversion and the like.

Sulfonated porphyrins have been incorporated as counter ions into conducting polymer structures.

One of the limitations possessed by previous porphyrin-containing polymers and polymerisable monomers has been that immobilisation or integration of the large porphyrin moiety in the polymer has significantly disrupted electronic communication within the polymer. This is reflected in the poor conductivity of these porphyrin-polymer films.

In addition, the integration of the porphyrin into the polymer chain has frequently changed the characteristics of the polymer for which it was chosen.

Furthermore the ability to polymerise known porphyrin-containing monomers can be limited.

The attachment of two or more selected polymerisable monomer units to the porphyrin and subsequent homopolymerisation, or copolymerisation of the monomer units has the potential to afford porphyrin cross-linked polymers in which the desired characteristics of both the polymer and the porphyrin are retained.

It is an object of the present invention to provide such porphyrin cross-linked polymers and thereby overcome or ameliorate at least one of the disadvantages of the prior art, or to at least provide a useful alternative.

### SUMMARY OF THE INVENTION

The present invention provides polymers in which the polymerised monomer units of the polymer are cross-linked by an electrofunctional unit.

Typically a pair or a quartet of polymerised monomer units of the polymer are cross-linked by the electrofunctional unit.

Cross-linked pairs or quartets of polymerisable monomer units useful in the preparation of the so called “electrofunctional unit cross-linked polymers” of the invention are also provided.

In particular the invention provides electrofunctional unit cross-linked polymers wherein the polymers are prepared as copolymers as opposed to homopolymers.

Amongst other properties, these copolymers demonstrate enhanced photovoltaic performance.

Throughout this specification the term “electrofunctional” is taken to refer to groups or units, which are adapted to donate or accept electrons, or possess inherent photovoltaic or chemical transport properties as exemplified by porphyrin.

Other electrofunctional units include tetranitrogen-containing macrocycles derived from the tetrapyrrolles porphin, chlorins and corrins as referred to in DE 42 42 676 A1.

According to a first aspect, the invention consists in a cross-linked pair of polymerisable monomer units having the structure:
Q-(L)ₙ-P-(L′)ₘ-Q′

where Q and Q′ are the polymerisable monomer units, P is an electrofunctional unit and L and L′ are linkers providing direct or indirect electronic communication between Q and P and between P and Q′, and wherein n=0, 1, 2 or 3 and m=0, 1, 2 or 3.

Preferably Q and Q′ are heteroaromatic rings of the general formula 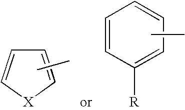
where R can be any suitable polymerisable or non-polymerisable functional group and X can be selected from S, NH or O. Suitable heteroaromatics include: thiophene, substituted thiophene, oligothiophene, furan, substituted furan, pyrrole and substituted pyrrole.

More preferably Q and Q′ are of molecular dimensions that permit polymerisation of the monomer units of the cross-linked polymerisable monomer units as a homopolymer.

Q and Q′ may be the same or different, preferably Q and Q′ are the same.

Preferably the linkers L and L′ are selected from the group comprising:

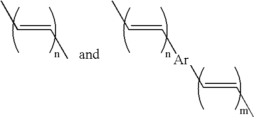

wherein n=0, 1, 2 or 3, m=0, 1, 2 or 3, and Ar is selected from the group comprising phenyl, naphthyl, polyaryl, heteroaryl, and ferrocenyl or similar metal sandwich complex.

L and L′ may be the same or different, preferably L and L′ are the same.

Preferably the electrofunctional unit P is selected from the group comprising: porphyrin, substituted porphyrin, phthalocyanine, substituted phthalocyanine or other tetranitrogen-containing macrocycle.

The electrofunctional unit P may or may not be coordinated to metals. Preferably the electrofunctional unit is coordinated to metal. Preferably the metal is zinc.

According to a second aspect, the invention consists in an electrofunctional unit cross-linked polymer comprising the structure:

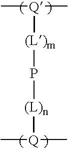

where P is the electrofunctional unit, Q and Q′ are monomer units of the polymer L and L′ are linkers providing direct or indirect electronic communication between Q and P and between P and Q′, and wherein n=0, 1, 2 or 3 and m=0, 1, 2 or 3.

The preferments of Q and Q′, L and L′, and P are the same as the preferments for the first aspect, excluding the preferment that Q and Q′ are of molecular dimensions that permit polymerisation of the monomer units of the cross-linked polymerisable monomer units as a homopolymer.

According to a third aspect, the invention consists in a cross-linked quartet of polymerisable monomer units having the structure:

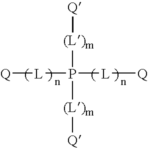

where P is an electrofunctional unit, Q and Q′ are the polymerisable monomer units, and L and L′ are linkers providing direct or indirect electronic communication between Q and P and between P and Q′, and wherein n=0, 1, 2 or 3 and m=0, 1, 2 or 3.

The preferments of Q and Q′, L and L′, and P are the same as the preferments for the first aspect.

According to a fourth aspect, the invention consists in an electrofunctional unit cross-linked polymer comprising the structure:

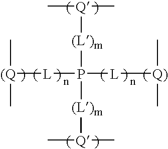

where P is the electrofunctional unit, Q and Q′ are monomer units of the polymer, L and L′ are linkers providing direct or indirect electronic communication between Q and P and between P and Q′, and wherein n=0, 1, 2 or 3 and m=0, 1, 2 or 3.

The preferments of Q and Q′, L and L′, and P are the same as the preferments for the first aspect, excluding the preferment that Q and Q′ are of molecular dimensions that permit polymerisation of the monomer units of the cross-linked polymerisable monomer units as a homopolymer.

According to a fifth aspect, the invention consists in an electrofunctional unit cross-linked polymer according to the second aspect of the invention wherein the polymer is a copolymer of the monomer units Q and Q′ and at least one other monomer unit. Preferably the other monomer unit is a substituted aromatic or heteroaromatic ring. More preferably the other monomer unit is selected from the group comprised of: benzene, substituted benzene, aniline, substituted aniline, thiophene, substituted thiophene, oligothiophene, furan, substituted furan, pyrrole and substituted pyrrole.

According to a sixth aspect, the invention consists in an electrofunctional unit cross-linked polymer according to the fourth aspect of the invention wherein the polymer is a copolymer of the monomer units Q and Q′ and at least one other monomer unit. Preferably the other monomer unit is a substituted aromatic or heteroaromatic ring. More preferably the other monomer unit is selected from the group comprised of: benzene, substituted benzene, aniline, substituted aniline, thiophene, substituted thiophene, oligothiophene, furan, substituted furan, pyrrole and substituted pyrrole.

According to a seventh aspect, the invention consists in a cross-linked pair of monomer units, cross-linked quartet of monomer units, polymer, or copolymer according to any one of the previous aspects further comprising a solubilising group. A preferred solubilising group is SO₃⁻.

According to an eighth aspect, the invention consists in an electrofunctional material including a base material and an electrofunctional unit cross-linked polymer or copolymer according to the second aspect or any one of the fourth to seventh aspects. Preferably the electrofunctional material is a photovoltaic material. Preferably the base material is textile, glass or metal.

According to a ninth aspect, the invention consists in a method of preparing a cross-linked pair of monomer units according to the first aspect, said method comprising the step of reacting a thiophenecarboxaldehyde with a dipyrrylmethane compound.

According to a tenth aspect, the invention consists in a method of forming a polymer according to any one of the second or fourth to seventh aspects comprising the steps of polymerising the monomer units of a cross-linked pair or quartet of polymerisable monomer units according to the first or third aspects, respectively. The polymerisation may be carried out by oxidation, which may be chemical or electrochemical. Preferably the polymerisation is electropolymerisation.

According to an eleventh aspect, the invention consists in a method of preparing an electrofunctional material comprising the steps of contacting a base material with a cross-linked pair or quartet of polymerisable monomer units according to the first or third aspects, respectively, and subsequently polymerising the monomer.

According to a twelfth aspect, the invention consists in a method of preparing an electrofunctional material according to the eleventh aspect further including the step of adding to the cross-linked pair or quartet of polymerisable monomer units at least one other monomer unit selected from the group comprised of: benzene, substituted benzene, aniline, substituted aniline, thiophene, substituted thiophene, oligothiophene, furan, substituted furan, pyrrole and substituted pyrrole.

According to a thirteenth aspect the invention consists in a method of light harvesting comprising the steps of applying a polymer or copolymer according to any one of the second or fourth to seventh aspects to a surface, applying light to the resultant surface, or exposing said surface to light, and capturing the resultant current.

According to a fourteenth aspect the invention consists in a method of light harvesting comprising the steps of applying one or more components selected from the group comprising a cross-linked pair or quartet of polymerisable monomer units of the first or third aspects, respectively, to a surface, polymerising such units in situ, optionally in the presence of another monomer, polymer or copolymer, applying light to the resultant surface, or exposing said surface to light, and capturing the resultant current. Suitable other monomers include benzene, substituted benzene, aniline, substituted aniline, thiophene, substituted thiophene, oligothiophene, furan, substituted furan, pyrrole and substituted pyrrole.

According to a fifteenth aspect the invention consists in a photovoltaic device incorporating a polymer according to any one of the second or fourth to seventh aspects.

Further embodiments of the invention are also envisaged which include the addition of components which exhibit electrochromism, thermochromism, optical activity, metal chelation, molecular recognition, chemical sensing, high anodic stability and liquid crystalline effects and also functionalities suitable for subsequent chemical derivatisation, for instance, to attach enzymes or other biological entities.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

### DESCRIPTION OF PREFERRED EMBODIMENT

Organic synthesis procedures and polymerisation protocols have been developed which allow the preparation of novel polymers in accordance with the invention. The polymers or copolymers are cross-linked by porphyrin either directly or via conjugated chains or aromatic groups. Such a structure enables interaction between the porphyrin moiety and the conducting polymer or copolymer with significantly reduced disruption of the polymer or copolymer. These porphyrin cross-linked polymers have enhanced photovoltaic and electron transfer performance compared to other porphyrin-containing structures and provide conducting polymers sensitive to chemicals capable of binding to the porphyrin or other tetrapyrrolic macrocycle.

The ability to form a polymer may be enhanced when the polymer is a homopolymer prepared by the polymerisation of selected cross-linked polymerisable monomer units or a copolymer as described herein. Whilst not wishing to be bound by theory it is believed the spacing of the porphyrin moiety reduces disruption of the polymer. It is believed this spacing may be achieved by selection of appropriately dimensioned monomer units when forming a homopolymer or formation of a copolymer of appropriate monomer ratio.

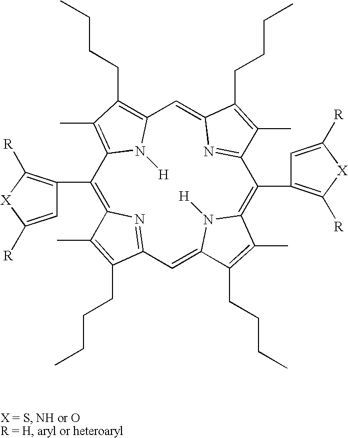

A cross-linked pair of polymerisable monomer units having thiophene (X=S, R=H) or terthiophene (X=S, R=thiophene) as the monomer units is shown above. Electrochemical or chemical polymerisation results in the aforementioned porphyrin cross-linked polymer. The polymer exhibits good photovoltaic performance due to the ability of the porphyrin moiety to harvest light and pass a hole to the conducting polymer “backbone”.

A synthetic methodology, illustrated in Scheme 1 has been developed which allows the synthesis of a wide variety of cross-linked pairs of polymerisable monomer units. Specifically I-III have been synthesised.

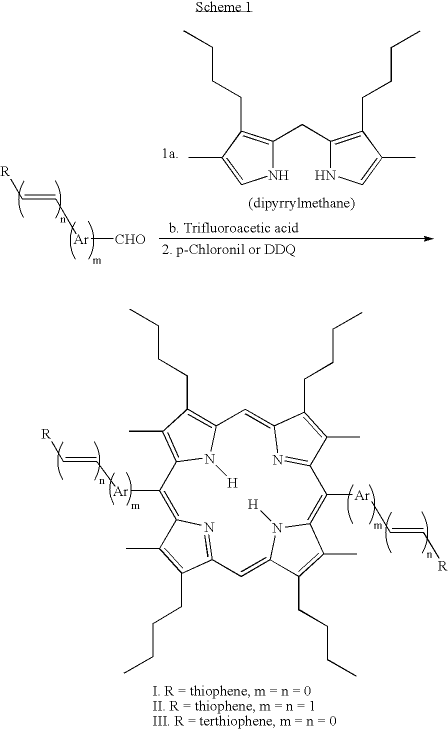

The terthiophene derivative (III) can be electrochemically polymerised to form a blue film, the CV of which is consistent with a conducting polymer.

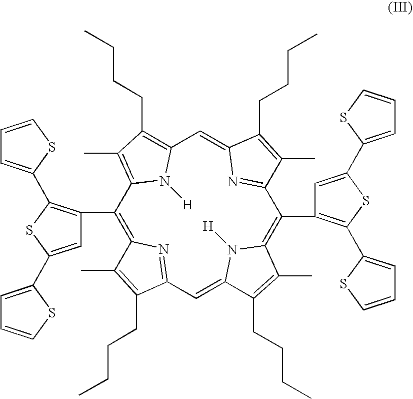

Notwithstanding this, these materials are of greater interest when used in the preparation of porphyrin cross-linked copolymers. Thus, copolymers containing (III) and terthiophene (1:2 ratio confirmed using elemental analysis) have been produced. The polymers are electrofunctional and exhibit the UV-vis spectra expected for a conducting polymer. Copolymerisation of this porphyrin terthiophene not only with other thiophenes but also with pyrrole and its derivatives is permitted.

Similar chemistry can also occur with porphyrin thiophenes (I and II). Thus these materials are extremely valuable in the preparation of electrofunctional polymers and copolymers.

One of the great advantages of using the porphyrin moiety is the ability to ‘tune’ the electronic state of the porphyrin by insertion of appropriate metal atoms. Thus the copper and zinc derivatives, (Cu-III) and (Zn-III), have been prepared and shown to act as copolymer cross-liners with terthiophene in the manner indicated above.

Polymerisation by the electrochemical route is preferred as it provides more accurate in situ control of the energy injected into the polymerisation reaction. It has been demonstrated for polypyrroles and polyanilines that this can be used to advantage in manipulating and improving the properties of the resultant material.

A variety of porphyrin derivatives in accordance with the invention can be made utilising the chemistry and compounds outlined in Scheme 1. Thus, a cross-linked pair of polymerisable monomer units can be prepared as an extended porphyrin-thiophene structure (II) as can the shorter porphyrin-thiophene structure (IV) by extension of the thiophene aldehyde.

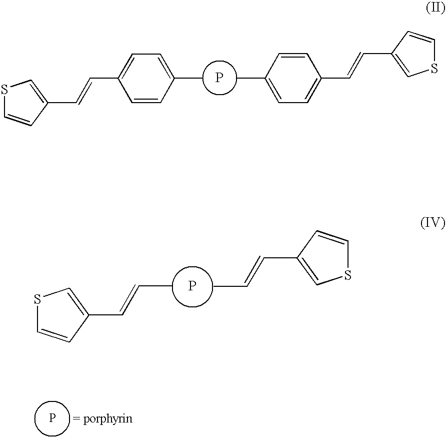

Identically substituted bithiophenes, terthiophenes and oligomers can also be made using this chemistry. Thus, bithiophene (V) and terthiophene (VI) are readily available from the corresponding aldehydes. The pyrrole or furan derivatives (VIIa,b), (VIIIa,b) and (IX) can similarly be prepared. 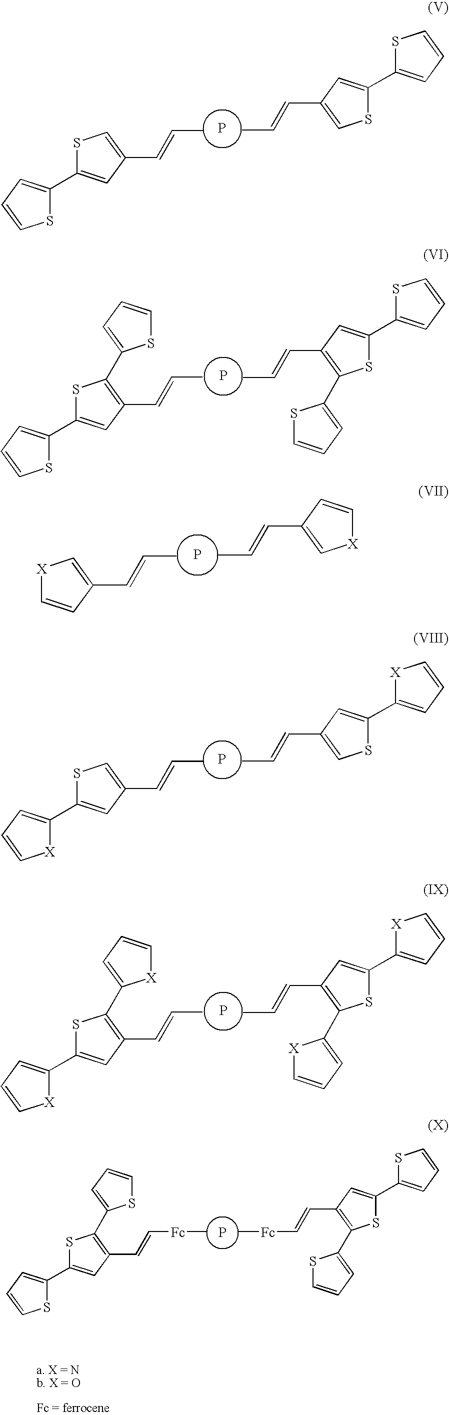
The aromatic rings in products such as (II) can be replaced with a variety of other useful derivatives such as ferrocene. Thus, the bisterthiophene-ferrocene-porphyrin (X) can be readily prepared from the appropriate terthiophene-ferrocene aldehyde using the procedure outlined in Scheme 1. This provides a way to introduce redox-active functionality into the cross-linked monomer units, in close proximity to the porphyrin moiety.

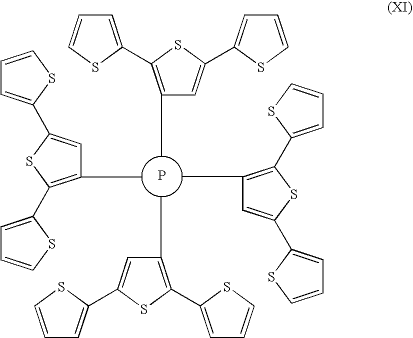

Replacing the dipyrrylmethane reactant in Scheme 1 with pyrrole provides a way to make a cross-linked quartet of polymerisable monomer units. The tetraterthiopheneporphyrin (XI) has been easily prepared this way and shown to undergo electrochemical polymerisation to give blue conducting polymer films.

Other photoactive functionalities can be exploited using this approach. For example, polypyridine functionalised terthiophenes (XII-XIV) are obtained from the reaction of terthiophene methylphosphonate with pyridine, bipyridine, or terpyridine aldehydes (Collis, G. E., Burrell, A. K., and Officer, D. L., Tetrahedron Letters, 2001, 42, 8733-8735). Complexation of these cross-linked monomer units with suitable metal ligand derivatives provides bisterthiophene metal complex cross-linked monomer units such as (XV). These cross-linked monomer units have the potential to provide light harvesting cross-linked conducting polymers, analogous to the porphyrin terthiophenes.

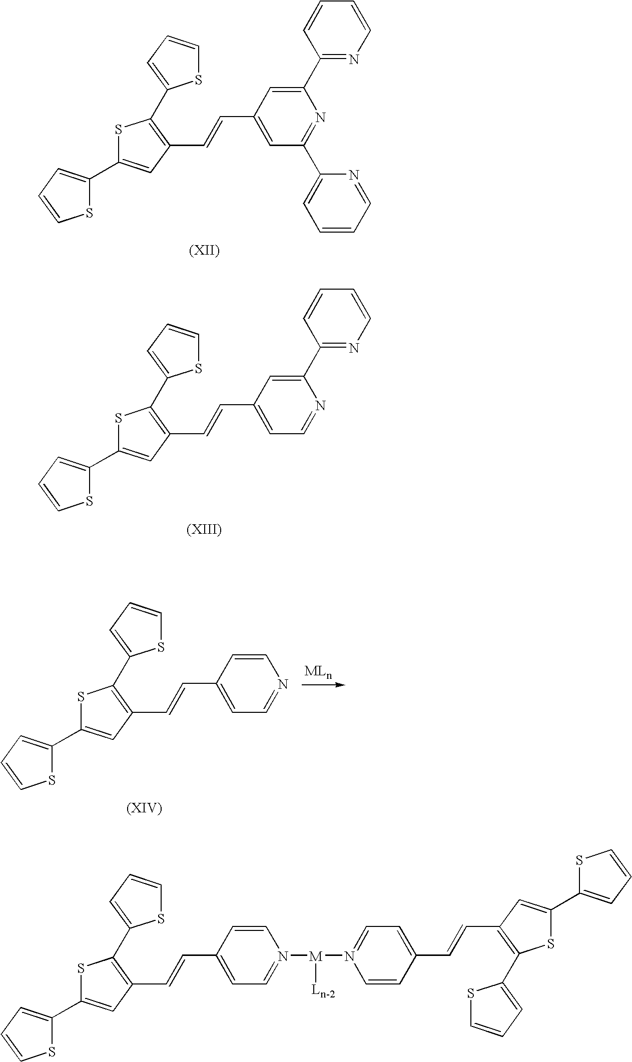

While the preferred embodiments herein are described in terms of cross-linked pairs or quartets of thiophene based monomer units, a person skilled in the art will appreciate all this chemistry is applicable to other heteroaromatic derivatives such as pyrrole and furan. For example, the heteroaromatic-porphyrin monomers (XVI) can be prepared and polymerised with thiophene, pyrrole and furan. Thus, oligomers such as (XVII) are available using the described methodologies.

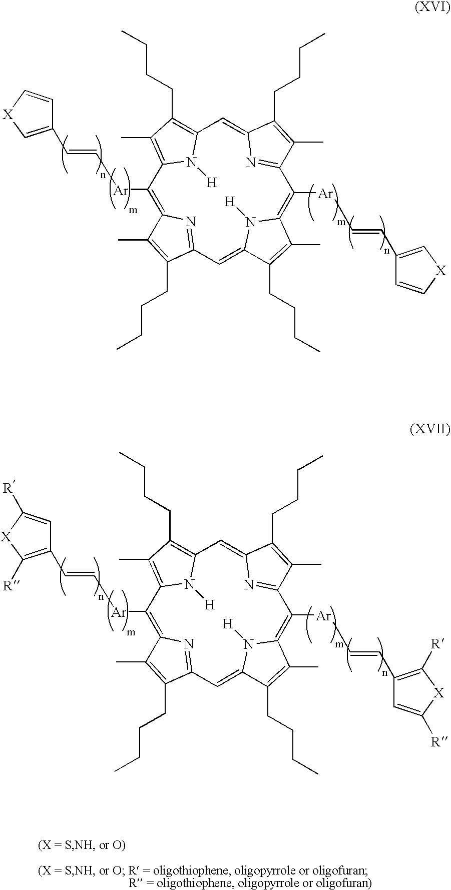

Many of the applications of conducting polymers with porphyrin groups attached in electronic communication benefit from production of polymers in “soluble” form. Such materials of the invention are then more amenable to subsequent processing as coatings or for device fabrication. Electro-hydrodynamic processing methods allow either colloids or truly soluble polymers to be produced if desired. These processing methods can be used in the production of colloidal forms, nanoparticles or nanofibres of the polymers of the invention. Alternatively soluble forms of the photoactive polymers can be prepared by forming copolymers with monomers such as (XVIII), 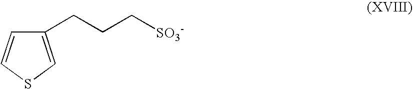
or by using polyelectrolytes as counterions to induce solubility.

A preferred application of the polymers, and in particular the copolymers, of the invention is in the production of photovoltaic materials, and in particular textiles. Chemical polymerisation directly onto substrates is achieved by dipping the substrate in a monomer of the invention followed by exposure to an oxidant. This process is applicable to either conductive or non conductive substrates including cloths, glass, or other structural materials.

Preferred embodiments will now be described with reference to the following figures and examples.

### FIGURES

FIG. 1 Cyclic voltammogram of monomer III (a); terphiophene (b); and diphenylporphyrin (c) at a platinum disk electrode. Solution: III or terthiophene or diphenylporphyrin (10 mM)/TBAP (0.1M)/DCM. Scan rate: 100 mV⁻¹.

FIG. 2 Potentiodynamic growth of the copolymer, poly(III-co-TTh), at a platinum disk electrode in III (5 mM/TTh (5 mM)/TBAP (0.1M)/DCM. Range: −1.0 to +1.0V. Scan rate: 100 mVs⁻¹.

FIG. 3 Post-polymerisation cyclic voltammogram of poly(iii-coTTh) in 0.1M TBAP/CH₃CN solution. Scan rate: 100 mVs⁻¹.

FIG. 4 UV-Vis spectra of poly(III-co-TTH) grown galvanostatically on an ITO coated glass electrode: (a) oxidised state; (b) reduced state.

FIG. 5 Scanning electron micrograph of poly(III-co-TTh)

FIG. 6 Comparison of the best energy conversion efficiency (ECE) results obtained from poly(III-co-TTh) with different monomer mole ratios of III:TTh in the polymerisation solution.

FIG. 7 UV-Vis spectra of monomer III with or without zinc incorporated: (a) free base and (b) zinc coordinated. Concentration of monomer I was 0.04 mM in DCM.

FIG. 8 Energy conversion efficiency (ECE) of poly(III-co-TTh): (a) free base, (b) zinc soaked.

### EXAMPLES

In the Examples, ¹H nuclear magnetic resonance (NMR) spectra were obtained at 270.19 MHz using a JEOL JMN-GX270 FT-NMR Spectrometer with Tecmag Libra upgrade, and at 400.132 MHz using Bruker 400 Avance running X-WIN-NMR software. The chemical shifts are relative to TMS or to the residual protium in deuterated solvents (CDCl₃, 7.25 ppm; pyridine-d₅, 7.00, 7.35, 8.50 ppm; DMSO-d₆, 2.50 ppm; MeOD-d₄, 3.35 ppm) when TMS is not present. ¹³C NMR chemical shifts are relative to CDCl₃ (77.0 ppm), pyridine-d₅ (123.4, 135.3, 149.8 ppm), MeOD-d₄, (49.0 ppm). Electronic absorption spectra were obtained using a Shimadzu UV-3101PC UV-VIS-NIR-Scanning Spectrophotometer. Mass spectra were recorded using a Varian VG70-250S double focusing Magnetic sector mass spectrometer. Samples analysed by fast atom bombardment (FAB) high resolution mass spectra (HRMS) were supported in a p-nitrobenzyl alcohol matrix and the data put through VG-OPUS software to give ±5 ppm error formulations on molecular ions. Major fragmentations are given as percentages relative to the base peak intensity. Chromatography was carried out using Silica (0.032-0.063 mm, Merck Kieselgel 60). Thin layer chromatography was performed using precoated silica plates (Merck Kieselgel 60F₂₅₄).

Chromatography solvents used in the Examples were laboratory grade. Water was purified by reverse osmosis. All other solvents used were AR grade unless otherwise stated. Iodine was sourced from M & B, and was resublimed to >99.8% purity. Na₂S₂O₃.5H₂O was sourced from BDH and was GP grade. 3-Thiophenecarboxaldehyde (98%) was sourced from Aldrich. 3′-Formyl-2,2′:5′,2″-terthiophene was prepared according to the procedure developed at Massey University (Collis, G. E., Burrell, A. K., and Officer, D. L., Tetrahedron Letters, 2001, 42, 8733-8735). The dipyrrylmethane was prepared according to the reported procedure (Sessler, J. L., Johnson, M. R., Creager, S. E. Fettinger, J. C. and Ibers, J. A., Journal of the American Chemical Society, 1990, 112, 9310-9329).

The reactions were carried out under an inert atmosphere and shielded from ambient light.

### Example 1

### Synthesis of 5,15-Bis(3′-thienyl)-2,8,12,18-tetra-n-butyl-3,7,13,17-tetramethylporphine (I)

3-Formylthiophene (30.6 mL, 0.349 mmol) and dipyrrylmethane (100 mg, 0.349 mmol) were dissolved in degassed anhydrous CH₂Cl₂ (35 mL) at room temperature. Then TFA (26.9 mL, 0.349 mmol, 1.0 equiv.) was added and the solution stirred under N₂. At the first sign of baseline material by TLC (≈15 minutes; silica gel, CH₂Cl₂) the reaction was quenched by the addition of DBU (52.2 mL, 0.349 mmol, 1.0 equiv.). Then p-chloranil (214 mg, 0.873 mmol, 2.5 equiv.) was added and the solution stirred for 4 h at room temperature. Next Et₃N (36 mL, 0.258 mmol) was added and reaction stirred vigorously for 1.5 h. Then excess Et₃N (0.723 mL, 5.190 mmol) was added and reaction stirred for 15 min (forms complex with p-Chloranil that is soluble in methanol). The product was precipitated out of solution with methanol, filtered and dried under high vacuum to give product (I) (69.3 mg, 53%) as a purple crystalline solid. ¹H NMR (270 MHz) □ −2.41 (br s, 2H, NH), 1.13 (t, 12H, J=7.3 Hz, CH₂CH₃), 1.78-1.90 (appt sex, 8H, J=7.6 Hz, CH₂CH₃), 2.13-2.28 (m, 8H, CH₂CH₂CH₃), 2.66 (s, 12H, CH₃), 4.03 (t, 8H, J=7.6 Hz, CH₂CH₂CH₂CH₃), 7.77 (br s, 6H, H thiophene), 10.26 (s, 2H, H 10, H 20).

¹³C NMR (68.1 MHz) □ 13.7, 14.3, 23.4, 26.5, 35.6, 97.0, 111.7, 124.4, 126.8, 132.6, 136.2, 141.3, 142.0, 143.1, 145.4. UV-vis (CH₂Cl₂): λₘₐₓ [nm] (ε×10⁻³) 408 (210), 507 (20), 542 (9.1), 574 (10), 627 (5.4), 674 (3.7), 725 (3.7). FAB-LRMS: m/z (%, assignment) 755 (100%, MH⁺). HRMS: Calcd for MH⁺ (C₄₈H₅₉N₄S₂): 755.4181, found: 755.4162.

### Example 2

### Synthesis of 5,15-Bis([2′,2″:5″,2″″-terthiophen]-3″-yl)-2,8,12,18-tetra-n-butyl-3,7,13,17-tetramethylporphine (III)

3′-Formyl-2,2′:5′,2″-terthiophene (96.5 mg, 349 μmol) and dipyrrylmethane (100 mg, 349 μmol) were dissolved in degassed dry CH₂Cl₂ (35 mL) at RT. TFA (26.9 μL, 349 μmol, 1.0 eq) was added and the solution stirred under N₂. At the first sign of baseline material by TLC (≈15 minutes; silica gel, CH₂Cl₂) the reaction was quenched by the adding DBU (52.2 μL 349 μmol, 1.0 eq). Then p-chloranil (214.5 mg, 873 μmol, 2.5 eq) was added and the solution stirred for 4 h at RT. Next Et₃N (36 μL, 258 μmol) was added and reaction stirred vigorously for 1 h. Excess Et₃N (723 μL, 5.190 mmol) was added and reaction stirred for 15 min. The product was then precipitated out of solution with MeOH, to give product (III) (76.9 mg, 41%) as a brownish-purple solid. ¹H NMR (400 MHz, CDCl₃, TMS): δ −2.309 (br d, 4H, [−2.304 (NH_{(αα or αβ)}), −2.314 (NH_{(αβ or αα)})]), 1.067 (t, 24H, ³J=7.3 Hz, CH₂CH₂CH₂CH₃), 1.65-1.78 (app sext, 16H, CH₂CH₂CH₂CH₃), 2.11-2.26 (app pent, 16H, CH₂CH₂CH₂CH₃), 2.898 (s, 24H, H_{Me-TBMP}), 3.90-4.10 (m, 16H, CH₂CH₂CH₂CH₃), 6.40-6.48 (m, 8H, H_{4′, 5′, thienyl A}), 6.753 (dd, 2H, ³J=3.4, ⁴J=1.5 Hz, H_{3′ (αβ or αα), thienyl A}), 6.786 (dd, 2H, ³J=3.4, ⁴J=1.5 Hz, H_{3′ (αα or αβ), thienyl A}), 7.12-7.15 (4H, [7.129 (dd, ³J=5.1, 3.6 Hz, H_{4″″ (αα or αβ)}), 7.136 (dd, ³J=5.1, 3.6 Hz, H_{4″″ (αβ or αα)}]), H_{thienyl C}), 7.29-7.31 [4H, [7.294 (dd, ³J=5.2 Hz, ⁴J=1.1 Hz, H_{5″″ (αα or αβ)}]), 7.304 (dd, ³J=5.2 Hz, ⁴J=1.1 Hz, H_{5″″ (αβ or αα)}]), H_{thienyl C}), 7.44-7.46 (4H, [7.448 (dd, ³J=3.7 Hz, ⁴J=1.1 Hz, H_{3″″ (αα or αβ)}) 7.458 (dd, ³J=3.7 Hz, ⁴J=1.1 Hz, H_{3″″ (αβ or αα)}), H_{thienyl C}], 7.630 (app d, 4H, [7.629 (H_{4″ (αα or αβ)}), 7.631 (H_{4″ (αβ or αα)})], H_{thienyl B}), 10.227 (app d, 4H, [10.224 (H_{10, 20 (αα or αβ)}), 10.230 (H_{10, 20 (αβ or αα)})], Hₘₑₛₒ). Assignments aided by COSY spectra. ¹³C NMR (101 MHz): δ 13.71, 13.72, 14.2, 23.3, 26.5, 35.6, 97.2, 108.6, 110.18, 110.21, 124.1, 124.2, 124.8, 124.9, 125.4, 126.2, 126.3, 128.1, 130.0, 130.1, 134.1, 134.2, 135.4, 135.5, 136.0, 136.1, 136.5, 137.2, 138.5, 141.9, 143.5, 145.4, 145.5. UV-vis (CH₂Cl₂): λₘₐₓ [nm] (ε×10⁻³) 415 (203), 511 (17.8), 546 (7.06), 577 (7.59), 630 (3.23). FAB-LRMS: m/z (%, assignment) 1083 (100, MH⁺). HRMS: Calcd for MH⁺ (C₆₄H₆₇N₄S₆): 1083.3690, found: 1083.3719.

### Example 3

### Synthesis of 5,15-Bis([2′,2″:5″,2″″-terthiophen]-3″-yl)-2,8,12,18-tetra-n-butyl-3,7,13,17-tetramethylporphyrinato zinc(II) (Zn-III)

A solution of Zn(OAc)₂.2H₂O (42.9 mg, 196 μmol, 1.2 eq) in MeOH (1.0 mL) was added to a solution of bisterthienylporphyrin (II) (177 mg, 163 μmol) in CHCl₃ (18 mL) with stirring at RT. The reaction was deemed complete by TLC (R_{f}=0.25, silica, CH₂Cl₂:hexane (1:2)) after 30 min. The crude product was precipitated with MeOH and the resulting solid was recrystallised from CH₂Cl₂/MeOH giving (Zn-III) (189 mg, 100%) as a brick-red powder. ¹H NMR (400 MHz, CDCl₃, TMS) δ 1.085 (t, 24H, ³J=7.3 Hz, CH₂CH₂CH₂CH₃), 1.68-1.79 (app sex, 16H, CH₂CH₂CH₂CH₃), 2.13-2.21 (app pent, 16H, CH₂CH₂CH₂CH₃), 2.915 (s, 24H, H_{Me-TBMP}), 3.90-4.05 (m, 16H, CH₂CH₂CH₂CH₃), 6.34-6.44 (m, 8H, H_{thienyl A}), 6.78-6.80 (m, 4H, H_{thienyl A}), 7.12-7.15 (4H, [7.135 (dd, ³J=5.1, 3.7 Hz, H_{4″″ (αα or αβ)}), 7.137 (dd, ³J=5.1, 3.7 Hz, H_{4″″ (αβ or αα)}]), H_{thienyl C}), 7.28-7.31 [4H, [7.293 (dd, ³J=5.3 Hz, ⁴J=1.2 Hz, H_{5″″ (αα or αβ)}), 7.298 (dd, ³J=5.3 Hz, ⁴J=1.2 Hz, H_{5″″ (αβ or αα)}]), H_{thienyl C}), 7.45-7.47 (4H, [7.459 (dd, ³J=3.6 Hz, ⁴J=1.3 Hz, H_{3″″ (αα or αβ)}), 7.463 (dd, ³J=3.6 Hz, ⁴J=1.3 Hz, H_{3″″ (αβ or αα)}), H_{thienyl C}], 7.674 (s, 2H, H_{4″″ (αα or αβ), thienyl B}), 7.696 (s, 2H, H_{4″″ (αβ or αα), thienyl B}), 10.188 (s, 4H, H_{10, 20, meso}). Assignments aided by COSY spectra. ¹³C NMR (101 MHz): δ 14.2, 14.3, 23.3, 26.5, 35.6, 97.8, 111.4, 111.5, 124.0, 124.1, 124.58, 124.63, 124.7, 125.4, 126.02, 126.09, 128.1, 130.4, 130.5, 133.86, 133.92, 135.5, 135.6, 136.7, 137.4, 137.89, 137.92, 140.1, 143.7, 146.84, 146.86, 148.1. UV-vis (CH₂Cl₂): λₘₐₓ [nm] (ε×10⁻³) 353 (63.2), 418 (336), 504 (4.51), 543 (21.6), 581 (13.4). FAB-LRMS: m/z (%, assignment) cluster at 1143-1151, 1144 (80, M⁺). HRMS: Calcd for M⁺ (C₆₄H₆₄S₆Zn): 1144.2747, found: 1144.2791.

### Example 4

### Synthesis of 5,15-Bis([2′,2″:5″,2″″-terthiophen]-3″-yl)-2,8,12,18-tetra-n-butyl-3,7,13,17-tetramethylporphyrinato copper(II) (Cu-III)

A solution of Cu(OAc)₂.H₂O (111 mg, 508 μmol, 1.2 eq) in MeOH (10.0 mL) was added to a refluxing solution of free-base (III) (500 mg, 461 μmol) in CHCl₃ (50 mL) with stirring under N₂ atmosphere. After 15 h, TLC analysis indicated that all of (III) had been metallated. The solvent was removed in vacuo and the residue was column chromatographed (silica, 37 mm_{dia}×90 mm, CH₂Cl₂:hexane (1:2)) collecting the major red coloured band. Recrystallisation from CH₂Cl₂:MeOH gave (Cu-III) (437.5 mg, 83%) as a purple solid. UV-vis (CH₂Cl₂): λₘₐₓ [nm] (ε×10⁻³) 414 (379), 536 (22.0), 574 (20.3). FAB-LRMS: m/z (%, assignment) cluster at 1142-1149, 1143 (90, M⁺). HRMS: Calcd for M⁺ (C₆₄H₆₄N₄CuS₆): 1143.2751, found: 1143.2753.

### Example 5

### Synthesis of 5,10,15,20-Tetrakis([2′,2″:5″,2″″-terthiophen]-3″-yl)porphine (XI)

3′-Formyl-2,2′:5′,2″-terthiophene (158 mg, 0.572 mmol) and pyrrole (39.65 μL, 0.572 mmol) were dissolved in degassed anhydrous CH₂Cl₂ (57 mL) under N₂ at room temperature. BF₃.OEt₂ (7.0 μL, 57 μmol, 0.1 eq) was added and the solution stirred in dark for 2 h. p-Chloranil (105 mg, 0.429 mmol, 0.75 eq) was added and the solution stirred at reflux temperature for 2 hours. Next excess Et₃N was added and the solvent removed under reduced pressure. The residue was subjected to column chromatography (80 mm×37 mm^{dia}, CH₂Cl₂:hexane (1:1)) collecting the first major porphyrin coloured band (R_{f}=0.15, CH₂Cl₂:hexane (1:1)). The product was then precipitated out from a solution of CH₂Cl₂ with MeOH, then filtered and dried under high vacuum to give product (XI) (62.4 mg, 34%) as a purple powder containing an inseparable mixture of four isomers by ¹H NMR. ¹H NMR (270 MHz) δ −2.52-2.44 (2H, [−2.47, br s, −2.50, br s] NH), 6.30-6.47 (m, 8H, H thiophene), 6.66-6.79 (m, 4H, H thiophene), 7.06-7.13 (m, 4H, H thiophene), 7.23-7.30 (m, 4H, H thiophene), 7.39-7.46 (m, 4H, H thiophene), 7.76-7.90 (4H [7.77 (s), 7.78 (s), 7.82 (s), 7.84 (s), 7.86 (s), 7.90 (s), H 4″]), 8.93-9.00 (m, 8H, β-pyrrolic). UV-vis (CH₂Cl₂): λₘₐₓ [nm] (ε×10⁻³) 252 (43), 357 (100), 426 (220), 525 (21), 561 (6.4), 596 (7.5), 654 (1.7). LRMS: m/z (%, assignment) 1295 (100%, MH⁺). HRMS: Calcd for MH⁺ (C₆₈H₃₉N₄S₁₂): 1294.9823, found: 1294.9798.

### Example 6

### Electro-Copolymerisation of III with Terthiophene

The cyclic voltammetry and electro-copolymerisation of a preferred cross-linked pair of polymerisable terthiophene monomer units, III prepared in Example 2, are herein described by way of example only. Cyclic voltammetry in dichloromethane, containing 0.1 M tetrabutylammonium perchlorate supporting electrolyte, revealed that co-monomer oxidation commenced at approximately 0.70V vs Ag/Ag⁺.

This oxidation process modifies the naked Pt surface somewhat as is evidenced by the presence of a crossover in the cyclic voltammogram. This is due to deposition of the oligomeric or polymeric product.

Chronopotentiograms were recorded using a Pt working electrode whilst applying a current density of 0.5 mA/cm². On Pt a steady state potential of ≈0.80V was generated. The fact that this potential did not increase during the polymerisation time indicates that a conductive polymer was deposited.

Chronoamperograms were also recorded using a Pt working electrode whilst applying a constant potential of 0.90V. After the initial transient, the current increased steadily as the conductive polymeric product was deposited on the electrode.

Elemental analysis of the copolymer electrodeposited from an equimolar mixture solution of III and terthiophene indicated that its composition was 1:2:4 of III:terthiophene:perchlorate counter-anion.

### Example 7

### Photovoltaic Performance

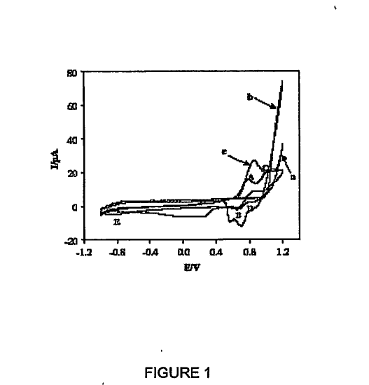

The photovoltaic performance of photoelectrochemical cells incorporating the conducting polymers or copolymers has been investigated. One possible embodiment of a photoelectrochemical cell is given in FIG. 1.

[image]

A halogen lamp was used as the white light source and usually an intensity of 500 W/m² was directed at the photoelectrochemical cell. Current-Voltage (I-V) curves were obtained from the photoelectrochemical cell in the dark or in the light by employing Linear Sweep Voltammetry. The I-V curves were then used to determine the Open Circuit Voltage (V_{oc}), the Short Circuit Current (I_{sc}), the Fill Factor, and the Energy Conversion Efficiency (ECE). Some results are shown in Table 1.

TABLE 1
Photovoltaic performance of photoelectrochemical cells
incorporating conducting polymers or copolymers.

| Type of | | | | |
|---|---|---|---|---|
| polymer or | | | | |
| copolymer | V_{oc} (mV) | I_{sc} (μA/cm²) | Fill Factor | ECE (%) |
| *Poly(MeTh) | 52 | 1.23 | 0.24 | 0.00005 |
| *Poly(BiTh) | 247 | 13.4 | 0.33 | 0.0034 |
| **Poly(BiTh) | 163 | 32.1 | 0.43 | 0.0071 |
| *Poly(TTh) | 187 | 13.1 | 0.35 | 0.0027 |
| **Poly(TTh) | 139 | 123.4 | 0.38 | 0.0205 |
| *Poly(TPP-TTh- | 185 | 15.9 | 0.28 | 0.0026 |
| co-TTh) | | | | |
| **Poly(TPP- | 169 | 27.9 | 0.35 | 0.0052 |
| TTh-co-TTh) | | | | |
| *Poly(TPP- | 218 | 4.74 | 0.30 | 0.0010 |
| TTh-co-BiTh) | | | | |
| **Poly(TPP- | 174 | 17.9 | 0.32 | 0.0031 |
| TTh-co-BiTh) | | | | |
| **Poly(BisTTh) | 195 | 229.0 | 0.38 | 0.035 |
| **Poly(BisTTh- | 212 | 475.0 | 0.35 | 0.069 |
| co-TTh) | | | | |
| **Poly(III-co- | 246 | 581.0 | 0.31 | 0.090 |
| TTh) | | | | |
| **Poly(III-co- | 185 | 881.0 | 0.36 | 0.116 |
| TTh) with Zn | | | | |
| coordinated | | | | |

Notes.
*solid polymer electrolyte.
**liquid electrolyte

Table 1 shows that the copolymer of III with terthiophene (TTh), Poly(III-co-TTh), gave the best results and that zinc coordination to the porphyrin moiety enhanced the photovoltaic performance.

### Example 8

### Photoelectrochemical Cell

The enhanced photovoltaic performance of Poly(III-co-TTh) was further investigated.

The porphyrin cross-linked copolymer poly(III-co-TTh) was prepared and incorporated into photoelectrochemical cells and tested for photovoltaic responses.

The effect of zinc incorporation into the porphrin cavity was also investigated.

Reagents and Materials

trans-5,15-Bis([2,2′;5′,2″-terthiophene]-3′yl)-2,8,12,18-tetra-n-butyl-3,7,13,17 tetramethylporphyrin (III) was synthesised as described in Example 2. 2,2′;5′2″-Terthiophene (TTh) (Aldrich), tetrabutylammonium perchlorate (TBAP, Fluka), iodine (Univar, Ajax or Aldrich 99.8%), methanol (Univar, Ajax), acetonitrile (ACN, Univar, Ajax), dichloromethane (DCM, Univar, Ajax), isopropanol (Univar, Ajax), tetrapropylammonium iodide (Aldrich, >=98%), ethylene carbonate (Aldrich 99%), zinc acetate (Fluka), propylene carbonate (Aldrich, 99%). ITO coated glass (=<10 Ωsq⁻¹) was purchased from Delta Technologies Limited (USA), cut into required sizes, washed with liquid detergent, rinsed thoroughly with Milli-Q water followed by isopropanol, and allowed to dry. Before coating with polymers, the ITO coated glass was treated in an UVO-cleaner (Model No. 42-220, Jelight Co. Inc., USA). Liquid electrolyte was prepared by dissolving I₂ (60 mM) and tetrapropylammonium iodide (500 mM) in ethylene carbonate/propylene carbonate (1:1 by weight). A thin layer of platinum was sputter coated onto ITO coated glass using a Dynavac Magnetron Sputter Coater Model SC100MS. The sputtering was performed at a current of 50 mA and Ar pressure of 2×10⁻³ mbar. A Pt thickness of 10 Å was sputter coated. These samples were used as counter electrodes to the polymer coated ITO coated glass.

Equipment and Methods

Electrosynthesis and testing of copolymers were achieved by using an electrochemical hardware system comprising of an EG&G PAR 363 Potentiostat/Galvanostat, a Bioanalytical Systems CV27 Voltammograph, a MacLab 400 with Chart v 3.5.7/EChem v 1.3.2 software (ADInstruments), and a Macintosh computer. A three-electrode electrochemical cell was used which comprised of a working electrode platinum disc or ITO coated glass or these substrates with polymer coatings on them), a platinum mesh auxiliary electrode and a Ag/Ag+ reference electrode with salt bridge. Copolymer samples were also subjected to elemental analysis (The Campbell Microanalytical Laboratory, Otago University, New Zealand). UV-Vis spectra of copolymer (oxidized and reduced states) were obtained using a Shimadzu UV1601 spectrophotometer and scanning over the range of 300-1100 nm. Scanning electron microscopy (SEM) examination was carried out on the copolymer films (solution side) using a Leica-stereo SS 440 Microscope. Conductivity measurement was carried out using a four-point probe connected to a HP34401A multimeter and constant-current source system (EG&G PAR 363 Potentiostat/Galvanostat). The electrochemically prepared polymers were tested using freshly prepared films (7-33 μm thick, using a digital micrometer (Mitutoyo, Japan)). Photovoltaic device testing was done using a halogen lamp (SoLux MR-16 from Wiko Ltd.) and a set-up comprising of a Macintosh computer/MacLab 400 with EChem v 1.3.2 software (ADInstrument)/CV27 Voltammograph (Bioanalytical Systems) to obtain the current-voltage (I-V) curves. A light intensity of 500 Wm² was used.

Photovoltaic Device Fabrication and Testing

The copolymers were electrodeposited onto ITO coated glass and rinsed with acetonitrile and then allowed to dry. In general, the polymer or copolymer coatings were completely electro-reduced at −0.8V in 0.1M TBAP/DCM before being assembled as photovoltaic devices in order to obtain the higher open circuit voltage (V_{oc}) through decrease in the chemical potential of the polymer [2]. The device was assembled by sandwiching a liquid electrolyte between the copolymer coated ITO coated glass electrode and the Pt sputtered ITO coated glass electrode. This was done with a border of parafilm as spacer between these two electrodes. The photovoltaic devices were tested by linear sweep voltammetry (LSV). The open circuit voltage (V_{oc}) is given when the current is zero, and the short circuit current (I_{sc}) is given when the voltage is zero.

Results

Poly(III-co-TTh)

The electroactivity of the porphyrin cross-linked bisterthiophene III was initially investigated (FIG. 1(a)). On comparison with the CV of terthiophene alone (FIG. 1(b)) and a diphenylporphyrin analogue of III (FIG. 1(c)), it was found that III underwent two redox processes (peaks A/B and C/D) due to the porphyrin moiety. The electro-oxidation of the terthiophene moieties become apparent at potentials anodic of peak C in FIG. 1(a). Another reduction peak (labelled E in FIG. 1(a)) was due to the reduction of O₂ dissolved in the solution. The anodic upper limit was varied from 1.2 to 2.0V, but none of these conditions resulted in formation of a conductive, electroactive polymer film. In addition, a homopolymer film could not be obtained on the platinum electrode using either galvanostatic or potentiostatic methods. The inability of III to form a homopolymer under these conditions is probably due to steric hindrance, given the large size of the molecule. Therefore, the co-polymerisation of III with terthiophene (TTh) was considered.

Electrochemical Copolymerisation of Terthiophene with Monomer III

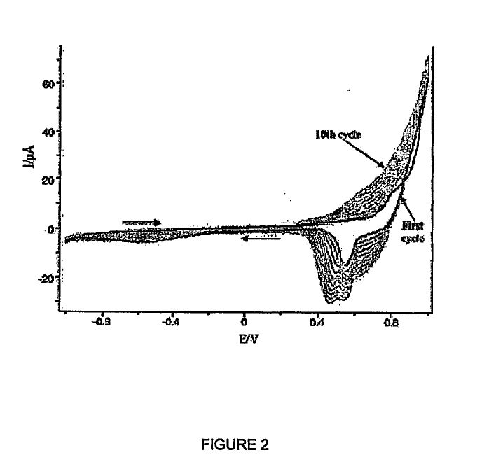

It was found that the copolymer of III with TTh could be successfully electrodeposited onto the platinum electrode using either cyclic voltammetry (CV), constant potential, or constant current. Potentiodynamic growth of this copolymer was performed at 100 mVs⁻¹ over the potential range from −1.0 to +1.0V from the co-monomer solution containing III (5 mM/terthiophene (5 mM)/TBAP (0.1 M)/DCM. The cyclic voltammograms obtained during growth (FIG. 2) showed the expected increase in redox current with increasing number of cycles, indicative of conducting electroactive polymer growth. The growth of a copolymer from a monomer solution containing equimolar mixture of III and TTh was confirmed by elemental analysis (2.52% N from the porphyrin moiety, 16.48% S from the terthiophene moiety, and 6.30% Cl). The results suggest that this polymeric material has a ratio of one unit of III:two terthiophene units:four perchlorate counter-anions. This high loading is indicative of copolymer formation with one perchlorate counter-anion to every three thiophene units. For the remainder of the description this copolymer is referred to as poly(III-co-TTh). Based on the first cycle in FIG. 2, the potential chosen for potentiostatic growth of poly(III-co-TTh) was +0.90V. A chronoamperogram typical of conducting polymer growth was obtained; after the initial transient, the current increased steadily as the copolymer continued to grow, resulting in an increase in surface area. Galvanostatic growth of poly(III-co-TTh) was performed at a constant current density of 0.5 mAcm⁻². The chronopotentiogram obtained displayed an initial transient and then a decreasing potential as expected of conducting electroactive copolymer growth. After 10 min, the potential obtained during growth was +0.80V.

Post-Polymerization Cyclic Voltammetry of Poly(III-co-TTh)

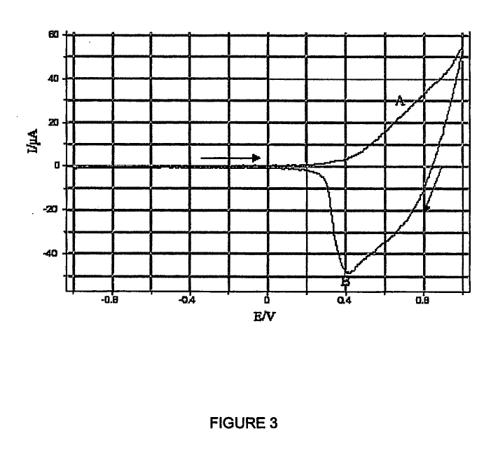

After the copolymer growth, the poly(III-co-TTh) modified platinum electrode was investigated both in 1.0M NaNO₃/H₂O and in 0.1M TBAP/ACN solutions using cyclic voltammetry. In aqueous solution containing 1M NaNO₃ supporting electrolyte, however, no polymer backbone peaks were observed, whereas a very stable redox couple (labelled A/B in FIG. 3) was observed in 0.1M TBAP/ACN. This is due to the fact that polythiophenes are, in general, more hydrophobic than polypyrroles and so electrochemical switching is less efficient in aqueous media [3]. The redox couple (labelled A/B) observed is attributed to oxidation/reduction of the copolymer back-bone. This cyclic voltammogram is not atypical of the CV of other substituted poly(terthiophene) films obtained by Cutler et al. [25,32].

UV-Vis Spectroscopy, Conductivity, and Scanning Electron Microscopy of Poly(III-co-TTh)

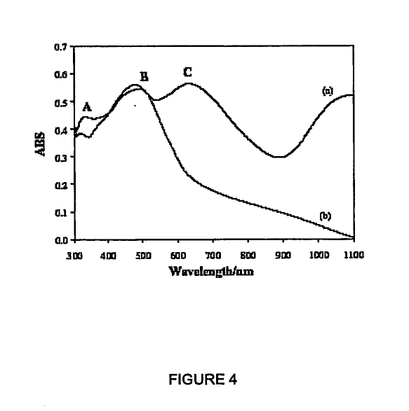

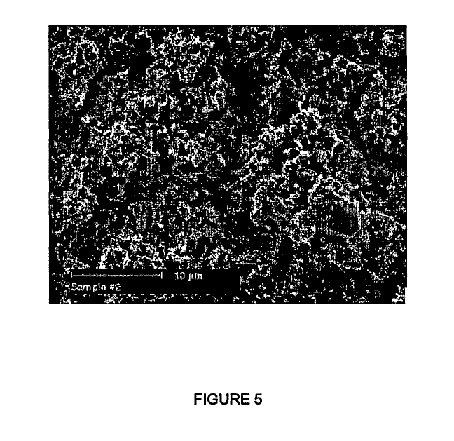

The UV-Vis spectra of the poly(III-co-TTh) film (electrodeposited onto ITO coated glass) were recorded (FIG. 4). In the oxidised state, the spectrum of poly(III-co-TTh) (FIG. 4(a)) exhibits a sharp peak (A) at 330 nm, two broad peaks at 505 nm (B) and 650 nm (C), and a free carrier tail that extends from 890 nm to longer wavelengths as expected of polythiophenes in the conductive state. The spectrum of its reduced state (FIG. 4(b)) shows that both the peak C at 650 nm and the free carrier tail are lost. This is in keeping with the loss of conductivity. On comparison with poly(terthiophene) [5], poly(III-co-TTh) displayed a stable absorption peak (B) at 505 nm for both oxidised and reduced states, which was not present in the spectrum of poly(terthiophene), and can be assigned to the absorption of the porphyrin moiety [34]. The conductivity of poly(III-co-TTh) was determined to be 0.24 S cm⁻¹, and the scanning electron micrograph (FIG. 5) of the solution side of poly(III-co-TTh) shows an open porous morphology that would be beneficial for photovoltaics in that the larger surface area should enhance the current obtainable from the photoelectrochemical cell.

Photovoltaic Testing of Devices Incorporating Poly(III-co-TTh)

Photoelectrochemical Cells

The copolymer, poly(III-co-TTh), was electrodeposited onto ITO coated glass electrodes instead of platinum disk electrodes, in order to fabricate them into photo-electrochemical cells. After poly(III-co-TTh) film growth, they were reduced at −0.8V in 0.10M TBAP/DCM solution before they were assembled into photovoltaic devices. All photoelectrochemical cells were assembled from poly(III-co-TTh) grown onto ITO

TABLE 2
Characteristics of photoelectrochemical cells incorporating
poly(III-co-TTh) grown by cyclic voltammetry from a 5:1 mole ratio for
1:2

| | Thickness | | | | |
|---|---|---|---|---|---|
| Number of | of film | | | | |
| Cycles | (μm) | ECE (%) | Fill factor | V_{oc} (mV) | I_{sc} (μA cm⁻²) |
| 5 | 0.910 | 0.041 | 0.29 | 217 | 317.8 |
| 10 | 1.284 | 0.042 | 0.27 | 214 | 368.0 |
| 15 | 1.458 | 0.055 | 0.30 | 212 | 435.3 |
| 20 | 1.865 | 0.055 | 0.28 | 239 | 403.8 |
| 25 | 2.420 | 0.043 | 0.29 | 242 | 303.8 |

coated glass by cyclic voltammetry between the potential limits −0.4 V/+1.2V at a scan rate of 100 mVs⁻¹. Initially, photovoltaic devices were made from copolymers grown by cyclic voltammetry from a monomer solution containing III (5 mM)/2 (1 mM)/TBAP (0.1 M)/DCM with potential limits from −0.4 to +1.2V at a scan rate of 100 mVs⁻¹. The thickness of polymer films was determined through controlling the number of cycles during growth (Table 2). Liquid electrolyte was used when fabricating photovoltaic devices. Table 2 summarizes the photovoltaic characteristics obtained from these completely reduced copolymers when fabricated into photoelectrochemical cells. These cells were tested with a 500 Wm⁻² halogen light source, and the testing area was 0.04 cm². The best cell incorporated a copolymer grown for 15 cycles by CV, and had the photovoltaic characteristics: V_{oc}=212 mV, I_{sc}=435 μAcm⁻², fill factor=0.30, and energy conversion efficiency (ECE)=0.055%.
Effect of Monomer Mole Ratios for III:TTh During Copolymer Growth on Photovoltaic Devices Response

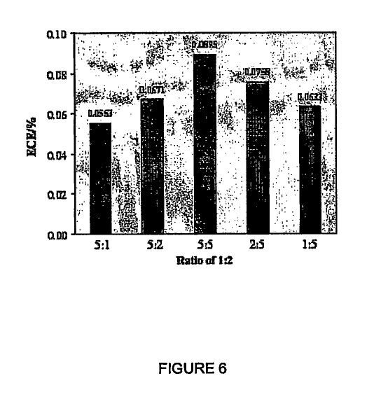

The copolymer composition was further optimised in order to obtain the best photovoltaic devices. A series of monomer mole ratios for III:TTh was investigated for the copolymer growth. The following co-monomer mole ratios of III:TTh were selected: 5:1, 5:2, 5:5, 2:5, and 1:5 mM. The deposited copolymers were also fully reduced at −0.8V before they were assembled into photoelectrochemical cells. Table 3 summarises the photovoltaic characteristic results obtained from these reduced copolymers. The results show that the energy conversion efficiency and short circuit current (I_{sc}) are affected by the thickness of the film and the monomer mole ratios during copolymer growth. FIG. 6 shows the best energy conversion efficiency results obtained from different monomer mole ratios for III:TTh. The best photovoltaic device (ECE=0.090%) incorporated a copolymer grown for 20 cycles by cyclic voltammetry at 100 mVs⁻¹, with potential limits −0.4 V/+1.2V, from a monomer solution containing III (5 mM)/TTh (5 mM)/TBAP (0.1 M)/DCM. This device had V_{oc}=246 mV, I_{sc}=581 μAcm⁻², fill factor=0.31, and ECE=0.090% under a halogen lamp intensity of 500 Wm². These results show that the monomer mole ratios used during poly(III-co-TTh) growth influences the photovoltaic responses. Table 3 also shows that both ECE and I_{sc} have the same trend with respect to the increase in polymer thickness (number of growth cycles). Whereas the other two characteristics, V_{oc} and fill factor appear independent of the thickness of the polymer film. These trends suggest that film thickness and short circuit current were the two dominant parameters influencing the energy conversion efficiency of these photoelectrochemical cells.

Photovoltaic Devices with Zinc Incorporated into the Porphyrin Cavity

The capture of sunlight by photosynthetic systems relies on the use of arrays of chlorophyll molecules.

TABLE 3
Characteristics of photoelectrochemical cells incorporating
poly(III-co-TTh) grown from different monomer mole ratios
for III:TTh

| Number of | ECE | | V_{oc} | I_{sc} |
|---|---|---|---|---|
| Growth Cycles | (5) | Fill Factor | (mV) | (μA cm⁻²) |
| For III:TTh = 5:1 | | | | |
| 5 | 0.041 | 0.29 | 217 | 317.8 |
| 10 | 0.042 | 0.27 | 214 | 368.0 |
| 15 | 0.055 | 0.30 | 212 | 435.3 |
| 20 | 0.055 | 0.28 | 239 | 403.8 |
| 25 | 0.043 | 0.29 | 242 | 303.8 |
| For III:TTh = 5:2 | | | | |
| 5 | 0.031 | 0.29 | 210 | 253.4 |
| 10 | 0.041 | 0.29 | 251 | 277.8 |
| 15 | 0.050 | 0.30 | 234 | 361.6 |
| 20 | 0.067 | 0.31 | 264 | 416.3 |
| 25 | 0.045 | 0.28 | 264 | 303.4 |
| For III:TTh = 5:5 | | | | |
| 5 | 0.032 | 0.29 | 242 | 224.4 |
| 10 | 0.048 | 0.29 | 236 | 348.1 |
| 15 | 0.060 | 0.32 | 235 | 405.6 |
| 20 | 0.090 | 0.31 | 246 | 581.0 |
| 25 | 0.064 | 0.32 | 266 | 383.8 |
| For III:TTh = 2:5 | | | | |
| 5 | 0.039 | 0.30 | 203 | 314.3 |
| 10 | 0.063 | 0.33 | 244 | 395.0 |
| 15 | 0.076 | 0.35 | 233 | 463.5 |
| 20 | 0.058 | 0.32 | 181 | 500.0 |
| 25 | 0.056 | 0.31 | 193 | 458.8 |
| For III:TTh = 1:5 | | | | |
| 5 | 0.034 | 0.33 | 193 | 263.5 |
| 10 | 0.053 | 0.34 | 195 | 398.8 |
| 15 | 0.063 | 0.31 | 181 | 568.8 |
| 20 | 0.061 | 0.34 | 179 | 504.6 |
| 25 | 0.041 | 0.32 | 184 | 344.3 |

Chlorophylls are magnesium-containing porphyrins. There has been over 40 years of work in porphyrin chemistry, attempting to emulate specific aspects of the light harvesting process, the majority of which involves the use of zinc-based porphyrin systems [6]. Zinc is the preferred metal for such work as it is easily introduced into porphyrins and zinc porphyrins are more stable than magnesium porphyrins.

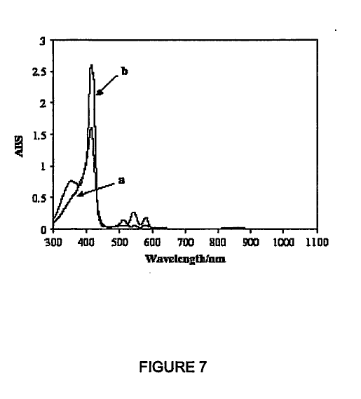

We have set out to demonstrate that the use of a porphyrin copolymer would enhance the light harvesting capability of a polythiophene-based photovoltaic device. Having demonstrated this effect with a free base porphyrin, the next step was to attempt to improve the light harvesting properties of the included porphyrin by complexing zinc into its cavity; given the extensive use of zinc porphyrins in molecular photonic devices [7]. Typical UV-visible spectra of monomer III with or without zinc incorporated were recorded at a monomer concentration of 0.04 mM in DCM (FIG. 7). They show that monomer III with zinc coordination has a higher light absorption than without zinc incorporated. Zinc in monomer III enhances light harvesting between 300-600 nm and this should be useful in promoting better photovoltaic performance. Further investigations of poly(III-co-TTh) were carried out by comparing the results with those obtained from samples of poly(III-co-TTh) films with zinc incorporated. In this study, all reduced poly(III-co-TTh) (5:5) modified ITO coated glass electrodes were exposed to a solution containing zinc acetate (0.001 M)+TBAP (0.1 M) in methanol for 2 days. These copolymer modified ITO coated glass electrodes were rinsed thoroughly with acetonitrile, and were allowed to dry.

TABLE 4
Characteristics of photoelectrochemical cells obtained from zinc-soaked
and free base poly(III-co-TTh) grown from a 5:5 mole ratio for III:TTh

| Number | ECE (%) | | Fill factor | | V_{oc} (mV) | | Isc (μA cm⁻²) | |
|---|---|---|---|---|---|---|---|---|
| of | | Zinc- | | Zinc- | | Zinc- | | Zinc- |
| Cycles | Free base | soaked | Free base | soaked | Free base | soaked | Free base | soaked |
| 5 | 0.032 | 0.069 | 0.29 | 0.35 | 242 | 210 | 224.4 | 487.5 |
| 10 | 0.048 | 0.100 | 0.29 | 0.39 | 236 | 216 | 348.1 | 615.0 |
| 15 | 0.060 | 0.116 | 0.32 | 0.36 | 23.5 | 185 | 405.6 | 881.3 |
| 20 | 0.090 | 0.113 | 0.31 | 0.34 | 246 | 202 | 581.0 | 797.5 |
| 25 | 0.064 | 0.079 | 0.32 | 0.31 | 266 | 198 | 383.8 | 626.8 |

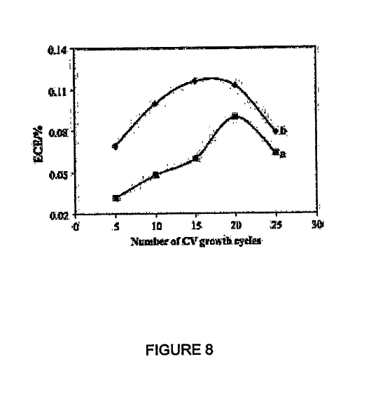

Table 4 summarises the photovoltaic characteristic results obtained from these reduced copolymers with and without being zinc-soaked. The results show that the values of ECE, fill factor, and I_{sc} all increased after the copolymer was zinc-soaked, while the value of V_{oc} decreased. FIG. 8 compares the ECE values for both zinc-soaked and free base poly(III-co-TTh). The best result was for the copolymer grown for 15 cycles where the ECE value was doubled (0.06-0.12%) compared to non-metallated samples.

Irrespective of copolymer thickness, the ECE values for poly(III-co-TTh) with zinc incorporated always showed increases relative to the free base copolymer. This may be due to the zinc coordinated into the porphyrin moiety, which would increase the quantum yield of the photovoltaic device. This is supported by the elemental analysis of zinc in the polymer, which shows that the mole ratio of zinc:porphyrin is 1:4. This suggests that possibly one out of four porphyrin sites is metallated.

### CONCLUSIONS

Copolymer deposits containing trans-5,15-bis([2′2″5″2″-terthiophene]-3′yl)-2,8,12,18-tetra-n-butyl-3,7,13,17-tetramethyl porphyrin (III) with 2,2′:5′2″-terthiophene (TTh), poly(III-co-TTh) were successfully electrosynthesised. In general, the copolymers had low conductivity, however, the UV-Vis spectra still showed the expected differences in absorbances between the fully oxidized (conducting) and fully reduced (semiconducting) states. Poly(III-co-TTh) contains a light harvesting moiety (porphyrin) cross-linking the polymer backbone. The monomer mole ratio for III:TTh during poly(III-co-TTh) growth had a great effect on the photovoltaic response (FIG. 2). The best mole ratio for III:TTh for photovoltaic devices is 1:1. This is due to the different percentages of III and TTh in the copolymer backbone produced from different monomer mole ratios during growth. Significant improvement in V_{oc} and I_{sc} as compared to the devices described by Yohannes et al. [1] from poly(3-methylthiophene) (V_{oc}=140 mV, I_{sc}=0.35 μAcm⁻²) have been obtained. Furthermore, comparison with our more recent work e.g. Cutler et al. etc [25,32], shows that the ECE and I_{sc} values of these devices are also significantly higher than those published for poly(terthiophene) and poly(nitrostyrylterthiophene). The trends in energy conversion efficiency versus polymer thickness are similar to those for the I_{sc} response (Table 3). This again confirms that the I_{sc} is the dominant parameter to affect ECE. The best device was made from this copolymer grown by cyclic voltammetry from the mole ratio of 1:1 for monomer III:TTh, and zinc-soaked before being assembled as a photovoltaic device. This device had V_{oc}=185 mV, I_{sc}=881 μAcm⁻² (more than 2500 fold improvement and at half the light intensity as compared to Yohannes et al. [1]), fill factor=0.36, energy conversion efficiency=0.12%.

Although the invention has been described with reference to particular embodiments, those persons skilled in the art will appreciate that variations and modifications may be made without departing from the scope of the invention.

### REFERENCES

[1] T. Yohannes, T. Solomon, O. Inganaes, Synth. Met. 82 (1996) 215.
[2] C. A. Cutler, A. K. Burrell, G. E. Collis, P. C. Dastoor, D. L. Officer, C. O. Too, G. G. Wallace, Synth. Met. 123 (2001) 225.
[3] J. Ding, W. E. Price, S. F. Ralph, G. G. Wallace, Synth. Met. 129 (2002) 67.
[4] C. A. Cutler, A. K. Burrell, D. L. Officer, C. O. Too, G. G. Wallace, Synth. Met. 128 (2001) 35.
[5] J. Chen, A. K. Burrell, G. E. Collis, D. L. Officer, G. F. Swiegers, C. O. Too, G. G. Wallace, Electrochim. Acta 47 (17) (2002) 2715.
[6] D. Gust, T. A. Moore, porphyrin handbook, in: K. M. Kadish, K. M. Smith, R. Guilard (Eds.), vol. 8, Academic Press, San Diego, Calif., 2000, p. 153.
[7] D. Holten, D. F. Bocian, J. S. Lindsey, Acc. Chem. Res. 35 (2002) 57.

1. A cross-linked pair of polymerizable monomer unit having the structure:
Q-(L)ₙ-P-(L′)ₘ-Q′,
where Q and Q′ are polymerizable units,
L and L′ are linkers providing direct or indirect electronic communication between P and Q and Q′, and
P is an electrofunctional unit,
wherein n=1, 2, or 3, and
wherein m=1, 2, or 3.

2. The cross-linked pair of polymerizable monomer unit according to claim 1, wherein Q and Q′ are substituted aromatic rings or heteroaromatic rings selected from the group consisting of:
substituted aromatic, aniline, substituted aniline, thiophene, substituted thiophene, oligothiophene, furan, substituted furan, pyrrole and substituted pyrrole.

3. The cross-linked pair of polymerizable monomer unit according to claim 1, wherein L and L′ are selected from the group consisting of: 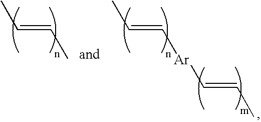
wherein n=1, 2, or 3,
wherein m=1, 2, or 3,
and Ar is selected from the group comprising phenyl, naphthyl, polyaryl, heteroaryl, and ferrocenyl.

4. The cross-linked pair of polymerizable monomer unit according to claim 1, where P is selected from the group consisting of:
porphyrin,
substituted porphyrin,
phthalocyanine,
substituted phthalocyanine, and
tetranitrogen-containing macrocycle.

5. The cross-linked pair of polymerizable monomer unit according to claim 1, wherein Q and Q′ are of molecular dimensions sufficient to permit polymerization of the monomer units of the cross-linked pair of polymerizable monomer units as a homopolymer.

6. An electrofunctional unit cross-linked polymer comprising the structure: 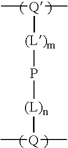
where Q and Q′ are monomer units of the polymer,
L and L′ are linkers providing direct or indirect electronic communication between
Q and P and between P and Q′, and
P is an electrofunctional unit, and
wherein n=0, 1, 2, or 3, m=0, 1, 2, or 3, and the polymer is a copolymer when m and n=0.

7. The electrofunctional unit cross-linked polymer according to claim 6 wherein Q and Q′ are substituted aromatic rings or heteroaromatic rings selected from the group consisting of:
substituted aromatic, aniline, substituted aniline, thiophene, substituted thiophene, oligothiophene, furan, substituted furan pyrrole and substituted pyrrole.

8. The electrofunctional unit cross-linked polymer according to claims 6, wherein L is selected from the group comprising: 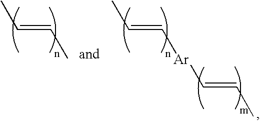
wherein n=0, 1, 2, or 3, m=0, 1, 2, or 3, and the polymer is a copolymer when m and n=0, and Ar is selected from the group comprising phenyl, naphthyl, polyaryl, heteroaryl, and ferrocenyl.

9. The electrofunctional unit cross-linked polymer according to claim 6, wherein P is selected from the group consisting of:
porphyrin,
substituted porphyrin,
phthalocyanine,
substituted phthalocyanine, and
tetranitrogen-containing macrocycle.

10. The electrofunctional unit cross-linked polymer according to claim 6, wherein the polymer is a copolymer of the monomer units Q and Q′ and at least one other monomer unit.

11. The electrofunctional unit cross-linked polymer according to claim 10, wherein the other monomer unit is a substituted aromatic or heteroaromatic ring.

12. The electrofunctional unit cross-linked polymer according to claim 11 wherein the other monomer unit is selected from the group consisting of:
substituted aromatic, aniline, substituted aniline, thiophene, substituted thiophene, oligothiophene, furan, substituted furan, pyrrole, and substituted pyrrole.

13. The electrofunctional unit cross-linked polymer according to claim 12, wherein the other monomer unit is terphiophene.

14. The electrofunctional unit cross-linked polymer according to claim 6, wherein the electrofunctional unit is coordinated with a metal.

15. The electrofunctional unit cross-linked polymer according to claim 14, wherein the metal is zinc.

16. The electrofunctional unit cross-linked polymer according to claim 6, wherein the polymer has been prepared by electropolymerization.

17. A cross-linked quartet of polymerizable monomer unit having the structure: 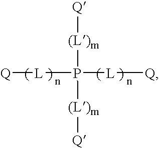
wherein:
Q and Q′ are the polymerizable monomer units,
L and L′ are linkers providing direct or indirect electronic communication between
P and Q and Q,
P is an electrofunctional unit,
n=1, 2, or 3, and
m=1, 2, or 3.

18. The cross-linked quartet of polymerizable monomer unit according to claim 17, wherein Q and Q′ are substituted aromatic rings or heteroaromatic rings selected from the group consisting of:
substituted aromatic, aniline, substituted aniline, thiophene, substituted thiophene, oligothiophene, furan, substituted furan, pyrrole and substituted pyrrole.

19. The cross-linked quartet of polymerizable monomer unit according to claim 17, wherein L is selected from the group consisting of: 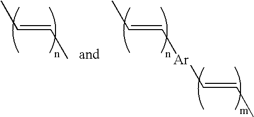
wherein n=1, 2, or 3, m=1, 2, or 3, and Ar is selected from the group consisting of phenyl, naphthyl, polyaryl, heteroaryl, and ferrocenyl.

20. The cross-linked quartet of polymerizable monomer unit according to claim 17 or claim 18 where P is selected from the group consisting of:
porphyrin,
substituted porphyrin,
phthalocyanine,
substituted phthalocyanine, and
tetranitrogen-containing macrocycle.

21. The cross-linked quartet of polymerizable monomer units according to any one of claims 17 to 20 wherein Q and Q′ are of molecular dimensions sufficient to permit polymerization of the monomer units of the cross-linked quartet of polymerizable monomer units as a homopolymer.

22. An electrofunctional unit cross-linked polymer comprising the structure: 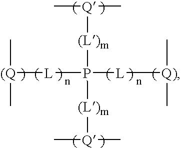
where Q and Q′ are monomer units of the polymer,
L and L′ are linkers providing direct or indirect electronic communication between
Q and P and between P and Q′, and
P is the electrofunctional unit,
wherein n=0, 1, 2, or 3, m=0, 1, 2, or 3, and the polymer is a copolymer when m and n=0.

23. The electrofunctional unit cross-linked polymer according to claim 22, wherein Q and Q′ are substituted aromatic rings or heteroaromatic rings selected from the group consisting of:
substituted aromatic, aniline, substituted aniline, thiophene, substituted thiophene, oligothiophene, furan, substituted furan, pyrrole, and substituted pyrrole.

24. The electrofunctional unit cross-linked polymer according to claim 22, wherein L is selected from the group consisting of: 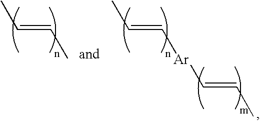
wherein n=0, 1, 2, or 3, m=0, 1, 2, or 3, the polymer is a copolymer when m and/or n=0, and Ar is selected from the group consisting of phenyl, naphthyl, polyaryl, heteroaryl, and ferrocenyl.

25. An electrofunctional unit cross-linked polymer according to claim 22 wherein P is selected from the group comprising:
porphyrin
substituted porphyrin
phthalocyanine
substituted phthalocyanine
tetranitrogen-containing macrocycle.

26. An electrofunctional unit cross-linked polymer according to claim 22 wherein the polymer is a copolymer of the monomer units Q and Q′ and at least one other monomer unit.

27. An electrofunctional unit cross-linked polymer according to claim 26 wherein the other monomer unit is a substituted aromatic or heteroaromatic ring.

28. An electrofunctional unit cross-linked polymer according to claim 27 wherein the other monomer unit is selected from the group comprising:
substituted aromatic, aniline, substituted aniline, thiophene, substituted thiophene, oligothiophene, furan, substituted furan, pyrrole and substituted pyrrole.

29. An electrofunctional unit cross-linked polymer according to claim 28 wherein the other monomer unit is terphiophene.

30. An electrofunctional unit cross-linked polymer according to claim 26, wherein the ratio of P to the at least one other monomer unit is 1:2.

31. An electrofunctional unit cross-linked polymer according to claim 22, wherein the electrofunctional unit is coordinated with metal.

32. An electrofunctional unit cross-linked polymer according to claim 31 wherein the metal is zinc.

33. An electrofunctional unit cross-linked polymer according to claim 22 wherein the polymer has been prepared by electropolymerisation.

34. A cross-linked pair of monomer units, cross-linked quartet of monomer units, polymer, or copolymer according to claim 1 further including a solubilising group.

35. A cross-linked pair of monomer units, cross-linked quartet of monomer units, polymer, or copolymer according to claim 34 wherein the solubilising group includes a SO₃⁻ moiety.

36. An electrofunctional material including a base material and an electrofunctional unit cross-linked polymer according to claim 6.

37. An electrofunctional material including a base material and a copolymer according to claim 6.

38. A method of preparing an electrofunctional material comprising the steps of treating a base material with a cross-linked pair or quartet of polymerisable monomer units according to claim 1 and subsequently polymerising the cross-linked pair or quartet of polymerisable monomer units.

39. A method of preparing an electrofunctional material according to claim 38, further including the step of adding at least one other monomer unit prior to polymerising.

40. A method according to claim 39 wherein the at least one other monomer unit is selected from the group comprising: substituted aromatic, aniline, substituted aniline, thiophene, substituted thiophene, oligothiophene, furan, substituted furan, pyrrole and substituted pyrrole.

41. An electrofunctional material according to claim 36 wherein the base material is textile, glass or metal.

42. A method according to claim 38, wherein the base material is textile, glass or metal.

43. A method according to claim 38, wherein the polymerising is by chemical or electrochemical oxidation.

44. Method of light harvesting comprising the steps of applying a polymer or copolymer according to claim 6, to a surface, applying light to the resultant surface, or exposing said surface to light, and capturing the resultant current.

45. Method of light harvesting comprising the steps of applying one or more components selected from the group comprising a cross-linked pair or quartet of polymerisable monomer units according to claim 1 to a surface, polymerising such units in situ, optionally in the presence of another monomer, polymer or copolymer, applying light to the resultant surface, or exposing said surface to light, and capturing the resultant current.

46. A method according to claim 44, wherein the another monomer is selected from the group consisting of benzene, substituted benzene, aniline, substituted aniline, thiophene, substituted thiophene, oligothiophene, furan, substituted furan, pyrrole and substituted pyrrole.

47. A photovoltaic device incorporating a polymer according to claim 6.

48-54. (canceled)
